(19) United States
(12) Plant Patent Application Publication
(10) Pub. No.: US 2005/0015835 P1
(43) Pub. Date: Jan. 20, 2005

(54) PAPAVER supperpoppy hybrid 'Heartbeat'

(50) PAPAVER HYBRID; HEARTBEAT

(76) Inventor: James Peter DeWelt, Atascadero, CA (US)

Correspondence Address: MRS. SANDRA M. WORTH, WATER MEADOW NURSERY CHERITON, NR. ALRESFORD, HAMPSHIRE, S024 OQB, GB

(21) Appl. No.: 10/619,411

(22) Filed: Jul. 16, 2003

(51) Int. Cl. A01H 5/00

(52) U.S. Cl. PLT/263

(57) ABSTRACT

The present Invention relates to a new and distinct cultivar of Papaver plant and hereafter referred to by the cultivar name as Papaver super poppy Heartbeat.

The new Papaver is a product of a planned breeding on Sep. 25^{th} 1997, by the inventor in Atascadero, in the County of San Louis Obispo. Calif. U.S.A.

The new Papaver is an inter-specific hybrid seedling, originated from a cross pollination of the following Papaver species:
Papaver atlanticum, Papaver: californicum, Papaver: rupifragrum, Papaver somniferum, and Papaver orientate.

The inventor selected the new Papaver in 1998 from the resulting progeny on the basis of its floriferousness, remarkable colouration, and its vigour. And named as Papaver superpoppy hybrid ‘Heartbeat’.

Asexual reproduction of the new hybrid, Papaver super poppy Heartbeat, by cuttings taken at Atascadero USA, has shown that the unique features of this new Papaver are stable and reproduces true to type in successive generations.

### SUMMERY OF THE INVENTION

Plants of the Poppy Papaver super poppy Heartbeat; have not been observed in all possible environmental conditions. The phenotype may vary somewhat with variations of environment such as temperature, light intensity, day length, and fertility level with out, however, any variance in genotype.

The following trials have been repeatedly observed and are determined to be the unique characteristics of ‘Heartbeat’. These characteristics in combination distinguish ‘Heartbeat’ as a new and distinct cultivar.

1. Upright habit, with thick supporting stems.

2. Dark, glossy green foliage

3. Large semi double purple/maroon flowers.

4. Thick, glossy flower petals, that with stand the rigors of Ultra Violet rays, rain & wind and therefore in flower for longer.

5. Vigorous growth habit.

The instant plant differs from one of the parent species (Papaver orientate) in the following ways.

1. Papaver Heartbeat plants are faster growing than Papaver orientate in all climate zones.

2. Papaver Heartbeat plants produce more flowers in total than Papaver orientate.

3. Papaver Heartbeat has a longer flowering season than plants Papaver orientate.

4. The flowers of Papaver Heartbeat have a distinct, much deeper purple/red, nearest colour 59A, 59B (RHS Colour chart) not seen in Papaver orientate or the parental species.
Comparisons:

Papaver super poppy ‘Heartbeat’ is a new hybrid. There are no known poppies to compare.

### BRIEF DESCRIPTION OF THE DRAWINGS

The accompanying coloured photographs illustrate the overall appearance of the new cultivar, showing the colours as true as it is reasonable possible to obtain in coloured reproductions of this type. Colours of the photographs may differ slightly from the colour values cited in the detailed botanical description, which more accurately describe the actual colours of the new cultivar. All photographs are of a mature plant (3 years old) of Papaver super poppy Heartbeat grown in garden soil.

FIG. 1. Top photograph: shows buds, stems, bracts, scales and a side view of an opening flower of Papaver superpoppy ‘Heartbeat’.

The lower photograph: shows a close up view of a typical flower of Papaver super poppy Heartbeat.

FIG. 2. Top photograph show a mature leaf of Papaver super poppy Heartbeat.

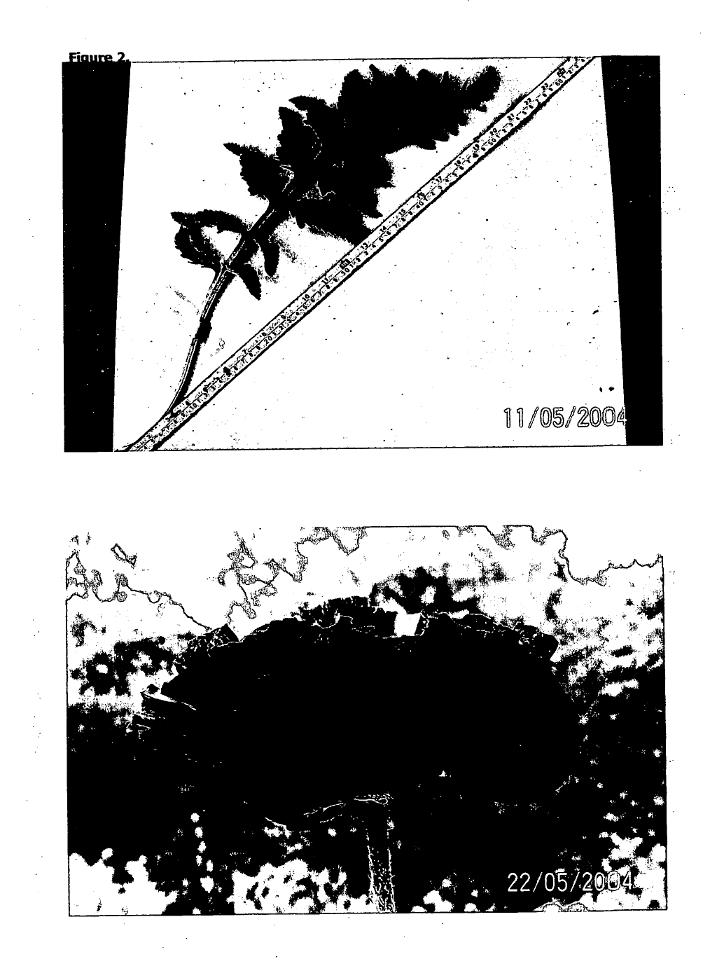

Lower photograph shows a close up of a side view of the stem scales & small bract, black vein lines and the opened bowl shaped flowers of Papaver superpoppy Heartbeat.

FIG. 3. This photograph shows (3) three stems, bracts, scales, and seed capsules of Papaver super poppy ‘Heartbeat’ placed on the right of the photograph.

On the left of the photograph is a picture of Papaver orientate ‘Royal Chocolate Distinction’ showing the seed capsule, stem, bracts & scale.

### DETAILED PLANT DESCRIPTION

In the following description, colour references are made to ‘The Royal Horticultural Society Colour Chart except where general terms of ordinary dictionary significance are used. Plants of the poppy Papaver super poppy Heartbeat were 3 years old and classed as mature and grown in garden beds with normal garden soil. Papaver super poppy Heartbeat is an inter-specific hybrid seedling from a sowing of seed on Sep. 25^{th} 1997 in the city of Atascadero, in the County of San Luis Obispo, Sate of Calif.

The breeding scheme was Papaver superpoppy hybrid×Papaver orientate of an unknown cultivar.
Parentage:

The female, or seed parent of this sowing being an inter-specific hybrid of the species Papaver atlanticum, Papaver californicum, Papaver rupifragrum, Papaver somniferum and Papaver orientate.

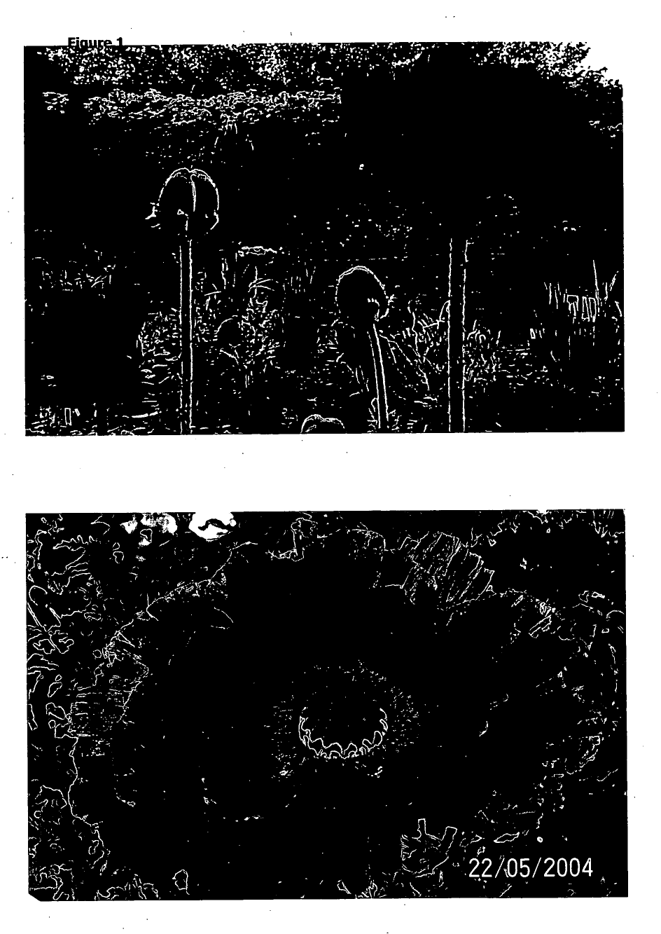

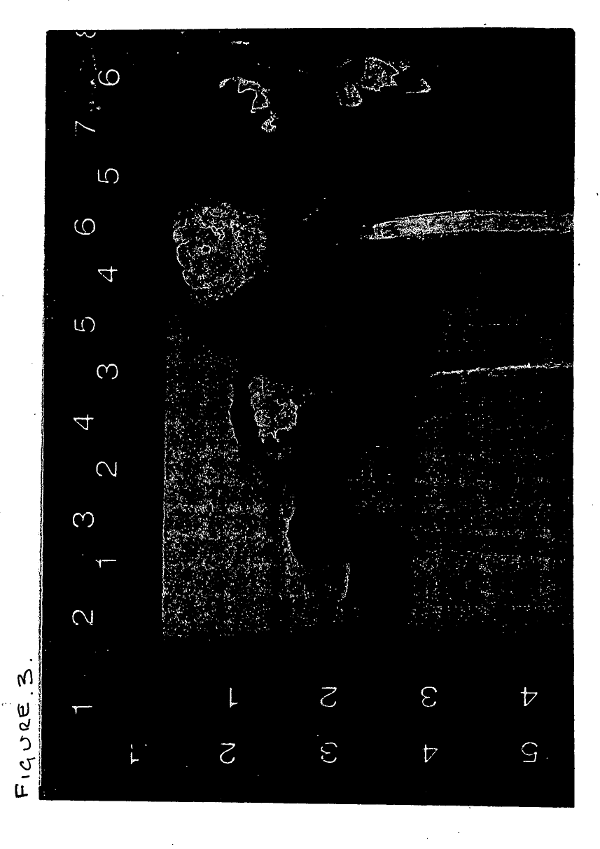

The male or pollen parent was an unnamed Papaver orientate seedling of unknown parentage. Trials were set up in both Atascadero. in the County of San Luis Obispo. Calif. U.S.A. from 1998 to 2001 and at the National Collection of Papaver U.K. in Cheriton. Hampshire. England. U.K. from 2000 to 2002. Both findings are listed below.
Propagation:
   Type.—Cuttings.
   Time to develop roots.—Cuttings harvested in October will develop roots in February in the Northern Hemisphere.
   Root description.—Fleshy.
   Crop time.—about 20 weeks are required to produce finished flowering plants for a 2 litre container.
Plant description:
   Form.—Robust hispid perennial flowering plant with upright, sturdy, erect, tall flowering stems, the foliage forming a symmetrical arching circle around the plant.
   Plant height.—about 36 inches.
   Plant diameter.—about 24 inches.
   Foliage description.—Leaves simple, generally symmetrical and long persisting.
   Arrangement.—Alternate & irregular.
   Shape.—Lanceolate, pinnately and deeply dissected.
   Length.—about 20 to 22 inches.
   Width.—about 7 inches.
   Leaf segment shape.—Lanceolate.
   Leaf segment apex.—Acuminate to acute.
   Leaf base.—Attenuate.
   Leaf segment margins.—Serrate to Serrulate.
   Leaves.—are sessile.
   Texture.—Glossy, slightly rogose; both upper & lower surfaces covered with pale grey to greenish white hairs between 1/16 to ⅛ inch in length.
   Colour.—Using the R.H.S. Colour charts.
   Young foliage upper surface.—137C.
   Young foliage lower surface.—137D.
   Mature foliage upper surface.—closest 137A to 137B.
   Veins.—145D.
   Mature foliage lower surface.—137C.
   Veins.—144B.
Flower description:
   Flower type & habit.—Large size, 6 petals, semi double flowers circular in shape, set with three outer overlapping petals and three more upright inside petals overlapping, the edges to all petals ruffled & slightly pleated, and one or two petals may have some serrated edges to the margins. Flowers upward & outward facing. Flowers not persistent. Freely flowering typically about 12 flowers and flowering buds at one time.
   Natural flowering season.—Late spring to early summer.
   Fragrance.—Not detected.
   Flowers longevity on the plant.—average 7 to 8 days before petals dropped.
Flower size:
   Diameter of flower.—5.5 to 7.5 inches.
   Depth (length) of flower.—about 3.5 inches.
   Bud.—(Before showing colour) oblong with domed bulbous top.
   Colour outside surface.—137C with maroon to purple dot markings, colour 71A.
   Length.—about 2.25 inches.
   Diameter.—about 1.25 inches.
   Texture.—Hispid covered with white, pale green to maroon red hairs.
   Length.—about 1/16 to ⅛^{th} inch.
   Corolla arrangement/appearance.—6 overlapping petals forming a bowl shape. Base indented to stem & bracts.
   Petal length.—about 4 inches.
   Petal width.—about 6.5 inches.
   Petal shape.—Orbicular to Reniform.
   Petal apex.—Obtuse.
   Petal base.—Truncate to Reniform.
   Petal margins.—Very fine crenulated edges, one or two petals may have incised margins, but this is not constant.
   Petal texture.—Strong, thick, satin smooth.
   Petal colour.—We believe this is a new colour break; the instant plant colour is deep Maroon with purple, & slight brown tones.
   Petal colour upper surface when opening.—Red Purple 59A./59B.
   Petal colour lower surface when opening.—closest 59A.
   Base.—with black venation lines extending to about 2 inches, over Colour 59A.
   Petal colour upper surface when opened.—Red Purple 59B with regular, oblong black blotch above the base: colour 46A.
   Petal colour lower surface when opened.—closest 59B.
   Lower surface base.—Black venation lines extending to about 2 inches, over colour 59B.
Peduncle/Flowering stems:
   Length.—about 36 inches.
   Diameter.—about ½ inch.
   Strength.—excellent, fully supporting the weight of the flowers, Thick, upright, self supporting, Texture hispid with pale grey to greenish white, horizontal hairs to lower stem and flat hairs to upper part of flowering stem. Diameter about ½. Inch
   Calyx/Sepal arrangement.—Typically 3.
   Calyx length.—about 2.25 inches.
   Calyx width.—about 1.5 inches.
   Sepal shape.—elliptic.
   Sepal apex.—mucronate.
   Sepal margin.—entire.
   Sepal margin colour.—149D.
   Sepal colour outer surface.—nearest 137C to 137D with maroon to purple dot markings, colour: nearest 71A.
   Colour inner surface.—nearest 149D.
   Texture.—hispid, covered with pale grey to red/purple hairs.
   Length of hairs.—⅛ to ¼ of an inch.
   Width of hair.—very fine.
   Colour outside surface.—137C with maroon to purple dot markings, colour 71A.
   Length.—about 2.25 inches.
   Diameter.—about 1.25 inches.
   Rate of opening.—about 3 weeks from visible bud to open flower.
   Peduncle/Stem colour.—137D.
   Peduncle leaves.—Smaller leaves attached to the stem at irregular intervals.
   Length of smaller leaves.—varying from 4.5 to 8 inches.
   Bracts.—Irregular, medium to small, with white hirsute edges to Apex and margins. Length 1.5 inches to 2.5 inches.
   Colour upper surface.—137D.
   Veins.—143B.
   Colour lower surface.—137C.
   Veins.—143B.
   Scales.—Obtuse with white hispid margins & apex. Scales not constant.
   Scale colour.—137D.
Reproduction organs:
   Stamens.—Quantity: Numerous, typically more than 200.
   Anther shape.—Basifixed.
   Anther Length.—about 3/16 inch.
   Anther colour.—202A.
   Pollen amount.—scarce.
   Pistils.—Quantity: One.
   Pistil length.—about ⅛^{th} inch.
   Stigma colour.—200A.
   Seed or poricidal capsule.—The instant plant had very large, attractive seed capsule adding to the uniqueness, (see FIG. 1. lower photo. & FIG. 3.) with pronounced indented markings on the top edge of the pericarp even when unripe and in flower.
   Colour when in flower.—nearest 142D.
   Length.—about 1.50.
   Diameter.—about 1.25 inches.
   Colour of seed capsule.—133D.
Seed description:
   Length.—1/16^{th} inch.
   Diameter.—1/16^{th} inch.
   Seed colour.—Dull dark brown.
   Disease resistance.—The instant plant has been noted to be free of pathogens common to Papaver.
   Weather tolerance.—The instant plants are tolerant to rain & wind and U.V. rays.
   Comparisons.—This instant plant is a new hybrid poppy plant, therefore there are no other poppies to compare.
   Scale colour.—137D.
Reproduction organs:
   Stamens.—Quantity: Numerous, typically more than 200.
   Anther shape.—Basifixed.
   Anther length.—about 3/16 inch.
   Anther colour.—202A.
   Pollen amount.—scarce.
   Pistils.—Quantity: One.
   Pistil length.—about ⅛^{th} inch.
   Stigma colour.—200A.
   Seed or poricidal capsule.—The instant plant had very large, attractive seed capsule adding to the uniqueness, (see FIG. 1. lower photo. & FIG. 3.) with pronounced indented markings on the top edge of the pericarp even when unripe and in flower.
   Colour when in flower.—nearest 142D.
   Length.—about 1.50.
   Diameter.—about 1.25 inches.
   Colour of seed capsule.—133D.
Seed description:
   Length.—1/16^{th} inch.
   Diameter.—1/16^{th} inch
   Seed colour.—Dull dark brown.
   Disease resistance.—The instant plant has been noted to be free of pathogens common to Papaver.
   Weather tolerance.—The instant plants are tolerant to rain & wind and U.V. rays.
   Comparisons.—This instant plant is a new hybrid poppy plant, therefore there are no other poppies to compare.

1. PAPAVER superpoppy hybrid ‘Heartbeat’ is a new and distinct cultivar of Papaver plant, as illustrated and described.
